(19) United States
(12) Patent Application Publication
(10) Pub. No.: US 2010/0106107 A1
(43) Pub. Date: Apr. 29, 2010

(54) Membrane envelop bandage for application of medicine or ointment

(76) Inventors: Caroline M. Nash, Middleburg, VA (US); Tania Woerner, Purcellville, VA (US)

Correspondence Address: Caroline Nash;Nash & Titus, LLC, 21402 Unison Road, Middleburg, VA, 20117, US

(21) Appl. No.: 12/589,337

(22) Filed: Oct. 22, 2009

Related U.S. Application Data
(60) Provisional application No. 61/197,097, filed on Oct. 23, 2008.

(51) Int. Cl. A61L 15/44; A61F 13/02

(52) U.S. Cl. 604/290; 604/307

(57) ABSTRACT

A membrane envelope bandage for treating an animal foot or hoof wherein the bandage is generally arcuate in shape and has a membrane envelope affixed to an upper surface of the bandage. The membrane envelope contains a medicine or treatment for applying to the bottom of an animal's hoof or foot. When the bandage is affixed to the bottom of an animal's foot or hoof, the medicine is released from the membrane envelope while the bandage protects the animal's foot or hoof and keeps the medicine from being contaminated.

This application claims priority of Ser. No. 61/197,097 filed Oct. 23, 2008.

### BACKGROUND OF THE INVENTION

1. Field of the Invention

The invention relates to a membrane envelop bandage for application of medicine to the bottom of a hoof. More particularly, the invention relates to a membrane envelop bandage containing a medicine or ointment that can be easily applied to a hoof of a horse or other hoofed animal and kept in place via reinforced adhesive covering.

2. Brief Description of Related Art

Hoof abscesses occur when bacteria get trapped between the sensitive laminae (the tissue layer that bonds the hoof capsule to the coffin bone) and the hoof wall or sole. The bacteria create exudate (pus) and this turns into a painful abscess. Hoof abscesses are a very common problem and are caused by a variety of conditions. The hoof can be weakened by a very wet environment or can crack due to very dry conditions. When the hoof is compromised, bacteria enter and travel into the deeper tissues of the foot. Other causes for hoof abscesses are a misplaced nail when the horse is shod or bruising of the foot from stomping to remove flies or working on hard surfaces.

Hoof abscesses may take several days to develop and typically the horse owner doesn't recognize the condition until the horse is acutely lame. Most horses are reluctant to bear weight on the involved foot making this a medical emergency. A veterinarian is brought in to diagnose the condition and treat the horse's hoof to provide pain relief and prevent the infection from worsening.

The veterinarian will localize the site of the abscess using a hoof tester and once the location is found, a hoof knife will be used to create a hole for the abscess to drain. Depending on the size of the abscess, this procedure may require removal of a substantial amount of hoof wall or sole. Occasionally, abscesses will rupture on their own either from the sole or at the hairline above the hoof or coronary band. After the abscess has been located and opened, the foot must be medicated to resolve the pain and prevent further infection. This requires application of a medicine and padding to protect the foot. The typical procedure for treating an abscess or other condition of a horse's hoof is to place an amount of medicated salve from a jar on a square gauze bandage and apply both salve and bandage directly to the sole of the hoof. While holding the gauze and salve in place with one hand, a layer of padding is placed around the foot and then an elasticized bandage such as 3M Vetwrap® is wrapped around the hoof to keep the medicine, gauze and padding in place. Then, while still holding the hoof up, a durable tape such as duct tape is wrapped over the top of the elasticized bandage to protect the bandage from wear when the horse walks on it. This whole procedure must be performed without letting the horse's foot contact the ground. Thus, the person wrapping the horse's foot must support the horse's weight and manage all the bandaging materials simultaneously. This can be a very difficult and dangerous procedure.

There is a product on the market today, Animalintex® that is commonly referred to as a “poultice pad” wherein a non-woven pad is impregnated with a dry poultice. The poultice is activated by soaking the pad in water after which it is applied directly to the sole of the foot. This product replaces the step of dipping the gauze bandage in medicated salve; however, the arduous task of applying the padding layer, the elasticized bandage and the durable outer tape still exists. There is also the problem of the wet poultice pad dripping water onto and through the elasticized bandage making it less capable of sticking to the horse's hoof.

There are a number of products on the market that cover or protect a horse's hoof such as boots (See U.S. Pat. Nos. 5,528,885; 6,560,951; 5,209,048; 5,272,857) nail-less shoes (See U.S. Pat. Nos. 5,224,549, 5,638,905), rubber pads (U.S. Pat. No. 5,105,892), and boots for applying lotion to the outer wall of the hoof with an absorbent inner lining. In the latter, the lotion is physically applied to the lining before putting the boot on the horse.

In one product, (U.S. Pat. No. 6,122,901) there is an adhesive bandage for an animal foot with a pretreated pad with medication. The pad is fabric and would not be suitable for pretreating with gels, liquids, salves and the like as they would ooze out of the fabric during packaging, storage or transport and contaminate the adhesives on the bandage rending the adhesive ineffective and non-sterile. The pad would only be useful for pre-treating with dry substances prior to packaging that would not leak out of the fabric pad. Any wet treatment would have to be applied immediately prior to treating an abscess presenting the same difficulties described above.

None of these devices are suitable and/or practical for applying medicine and bandage directly to the sole of the hoof. None of them contain a membrane envelop capable of enclosing a variety of medicines or treatments. None of them are suitable for preparing a bandage that can be packaged prior to shipping or sale that envelops a liquid, or dry substance such as salve, ointment, gel, paste, powder, crystalline substance or combinations thereof. None of these devices would permit same to be applied to the hoof via an enveloped application of a substance while still allowing for an adhesive attachment to the hoof in a simple procedure.

Therefore, it is an object of the present invention to overcome the shortcomings of the aforementioned devices and to provide a means for medicating the sole of a horse's hoof with a single bandage containing a medicine that is ready for application.

Another object of the present invention is to eliminate the need for multiple applications involving the application of a medicine on gauze, then a protective padding layer, then an elasticized bandage and finally a durable outer tape to protect the bandage from wear.

It is another object of the present invention to shorten the time it takes to apply medicine and bandaging to a horse's hoof.

It is another object of the present invention to make it safer for a caretaker to treat a horse's hoof with medicine and bandaging.

It is another object of the present invention to provide a bandage that is inexpensive and disposable so that it does not require cleaning after each application.

It is another object of the present invention to provide a bandage that is capable of being sterile.

It is another object of the present invention to provide a medicated bandage with an adhesive that will securely attach to the hoof wall.

It is another object of the present invention to allow for a novel delivery system of the medicine such that the medicine is not exposed until it is actually applied to the horse's foot, thus keeping the properties of the bandage such as the adhesive layer protected.

These and other objects will become apparent upon a further reading of the detailed description.

### SUMMARY OF THE INVENTION

What is required to meet the objects of the invention is a single use hoof bandage which incorporates a membrane envelop containing a medicine or treatment such as a salve, ointment, gel, paste, powder or crystalline substance or other liquid or dry substance, an absorbent protective layer of padding and an adhesive layer which adheres securely to the outside of the hoof wall while providing a durable outer covering.

### BRIEF DESCRIPTION OF THE FIGURES

FIG. 1a is a plan view of the bandage according to the present invention showing an inner surface of the bandage that contacts a horse's foot;

FIG. 1b is a plan view of the bandage showing an outer surface that contacts the ground;

FIG. 2a is a cross section view of a bandage according to one embodiment of the invention showing an adhesive and a peel away strip affixed to the bandage;

FIG. 2b is a cross section of a bandage according to another embodiment of the invention wherein the peel away strip covers the entire upper surface of the bandage;

FIG. 3 is a cross sectional view of the bandage according to another embodiment of the invention wherein the membrane envelop is attached directly to the outer sole member showing a partition inside the envelop; and

FIG. 4 is a perspective view of the bandage applied to a horse's foot.

### DETAILED DESCRIPTION

The outer perimeter of a horses hoof is generally rounded or arcuate in shape and more arcuate at the toe of the hoof than at the heal of the hoof.

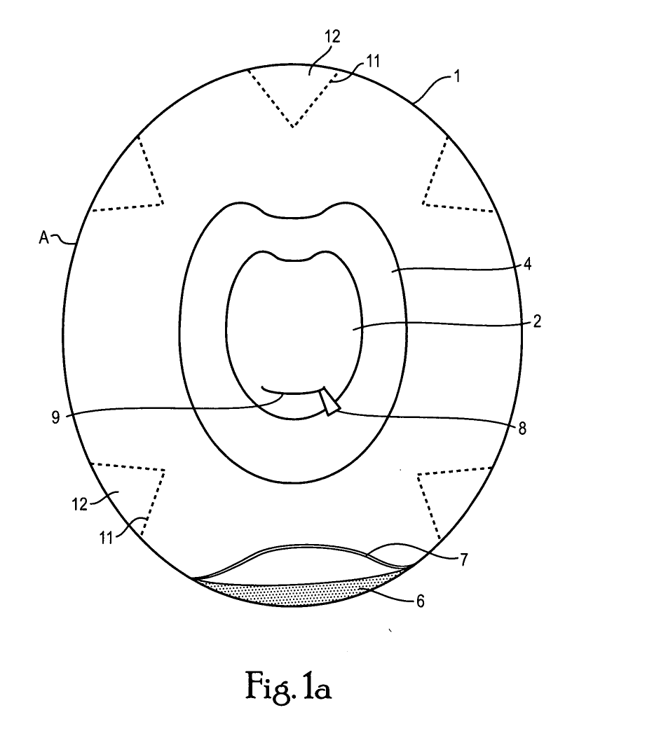

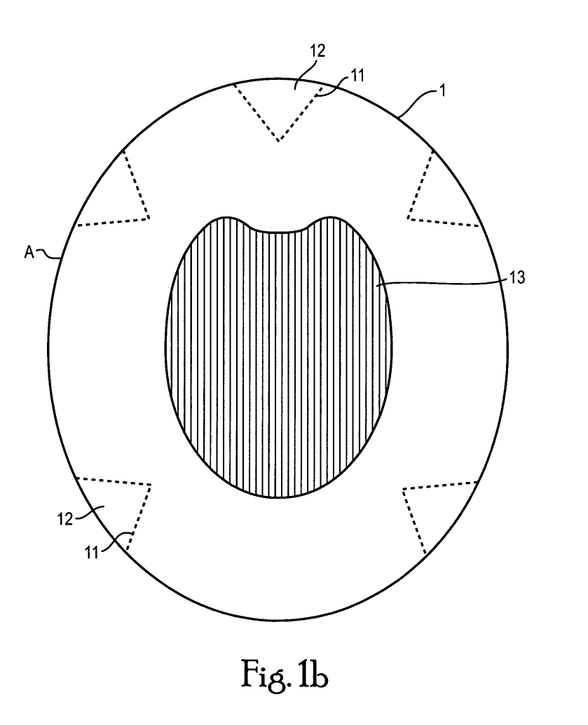

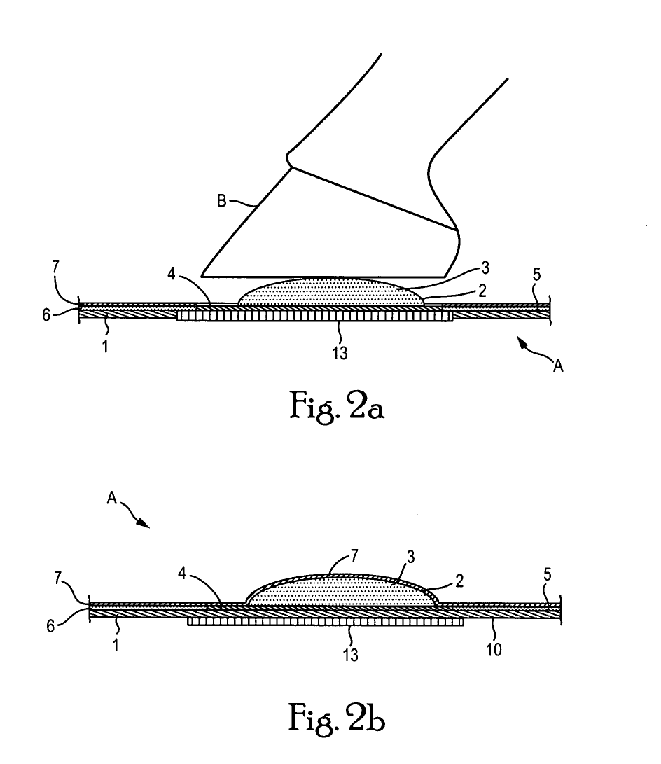

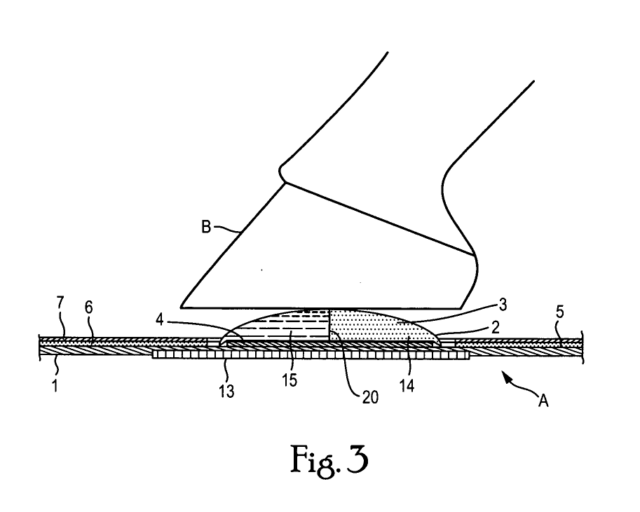

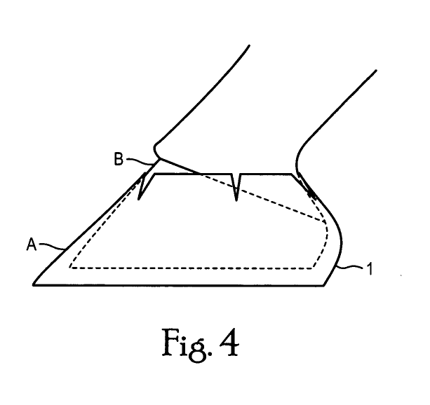

As shown in FIGS. 1a and 1b, the bandage A of the present invention has an outer sole member 1 that is flat and has a perimeter shape similar and perimeter size generally larger than the outer perimeter of a horses hoof. The outer sole member 1 comprises a protective covering extending outward from the membrane envelop 2 and absorbent layer 4 which is designed to be adhered to the outer wall of a hoof to keep the bandage A in place. As shown in FIG. 2a, on an inner surface 5 of the outer sole member 1 is an absorbent layer 4. Above the absorbent layer is a membrane envelop 2 containing a medicine 3 or treatment 3 for topical application. As shown in FIG. 3, the membrane envelop 2 may have a partition 20 for delivering a combination of powder 14 and liquid 15 to the horse's sole via the membrane envelop 2. The partition 20 may divide, within the membrane envelop, different ingredients for forming a medical or treatment composition or reaction product when the partition is broken. The topical application is encapsulated within a membrane envelop 2 that is above an absorbent layer 4 that is above an inner surface 5 of the outer sole member 1. The membrane envelop 2 holds the topical application medicine 3 next to the absorbent layer 4 to provide delivery of medicine to the sole of a horses hoof B. Optionally, an opening tab 8 is affixed to the membrane for a user to pull and open the membrane envelop at the tear line 9 to release the medicine held within the membrane (shown in FIG. 1a).

Optionally, the outer sole member 1 of the bandage A is elasticized for stretching over and around the hoof wall. Optionally, tear away notches 12 are defined by perforations 11. The tear away notches 12 can be removed so that the outer sole member 1 can lay flat upon the rounded outer perimeter of a horses outer hoof wall. As shown if FIG. 2a, in one embodiment of the invention, the outer sole member 1 has a layer of adhesive 6 disposed on a portion of the outer sole member for adhering the bandage A to the outer wall of a horse's hoof B. A removable peel away layer 7 may be disposed on the adhesive 6 that can be removed prior to application of the bandage to the horse's hoof. Of note is the feature that the peel away layer can be removed and the medicine is still safely contained in the membrane envelop until it is either intentionally punctured or broken. This feature protects the medicine from contamination prior to application.

As shown if FIG. 2b in another embodiment of the invention, the removable peel away layer 7 can have the same shape as the outer perimeter of the bandage and cover the entire inner surface of the bandage, including the outer sole member, absorbent layer and membrane envelop. This embodiment protects the entire bandage from contamination. Removing the peel away layer 7 would expose the bandage and the adhesive so that the bandage would be ready for placement and fastening to a horse's hoof.

In practice, the peel away layer 7 is removed and the bandage is applied to the bottom of a horse's hoof, by sticking the adhesive layer to the outer wall of the hoof. After the bandage A is applied to the bottom of a horse's hoof and adhered to the outer surface of the hoof, the horse is permitted to put weight on the bandage, thereby breaking the membrane envelop 2 and releasing the topical application of medicine 3 onto the surface of the horse's sole. Alternatively, the membrane envelop is broken or punctured before placement on the horse's hoof. This can be done with a sharp object. Or, as shown in FIG. 1 a, an opening tab 8 can be pulled to open the membrane envelop along the tear line 9. The absorbent layer 4 and outer sole member 1 keep the medicine 3 in place. The outer sole member 1 also acts to protect the horse's hoof. The absorbent layer 4 can be made of a material that will provide cushion to the sole of the hoof as well as absorb and hold the medicine in place. The cushioning effect of the absorbent layer will relieve pain and cooperate with the medicine to improve healing or treatment time and minimize fatigue.

The membrane envelop is a flexible thin membrane that will not be dissolved by the medicine and also be strong enough to hold the medicine from seepage when the bandage is not in use. The membrane envelope can be semi-permeable or non-permeable. The membrane envelop, when loaded with medicine or other treatment, must be weak enough to break open upon the pressure of the horse standing on the membrane or when the caretaker punctures it with a sharp object to thereby release the medicine or treatment. The membrane is preferably 0.01 mm to 2 mm thick. It may be a synthetic or natural material with the above characteristics. The membrane can be biodegradable. The membrane envelop should be understood to either be a complete envelop such as a pouch that holds the medicine inside entirely on its own or a single membrane sheet that when affixed to the outer sole member or absorbent layer forms an envelop with the outer sole member for holding medicine therein.

The membrane envelope can be placed on the outer sole member in a number of configurations. In one embodiment of the invention, the membrane envelope has the same as or slightly larger outer dimension than the absorbent layer. In another embodiment, the membrane envelope has an outer dimension that is smaller than the absorbent layer. The membrane envelope may also be removable or applied with adhesive. The membrane envelope can have a variety of shapes but it is preferred to have an arcuate shape to compliment the bottom surface of a horse's hoof

In still another embodiment, multiple membrane envelops are affixed to the absorbent layer.

The outer sole member 1 may be synthetic or natural material. It can be woven or non woven. It is preferred to be waterproof. The outer sole member may optionally have a reinforcement guard 13 as shown in FIG. 1b in the center of the bandage positioned to be directly beneath the bottom surface of the horse's hoof. The reinforcement guard 13 may be integral with the outer sole member, as shown in FIG. 2a, or may be an additional layer applied on an outer surface 10 of the outer sole member, as shown in FIG. 2b. The reinforcement guard will protect the bandage from damage and wear caused by the horse walking on the bandage. It is preferred that bandage A, outer sole member 1, reinforcement guard 13, the absorbent layer 4, and membrane envelop 2 all have an arcuate shape to compliment the bottom surface of a horse's hoof.

The bandage can be available in different sizes to accommodate different sized hooves such as, for example, a small circumference (3-4″), medium (4-6″), large (6-8″) or extra large (8-12″).

The absorbent layer 4 may be woven or non-woven, natural or synthetic material. Examples of suitable materials include but are not limited to cotton, gauze, felt or cellulose. The absorbent layer may be completely covered by the membrane envelop as shown in FIG. 3 or may be larger in diameter than the membrane pad as shown in FIG. 2a.

The adhesive layer 6 must be sufficiently strong to hold the bandage in place when the horse is standing or walking. However, it must also be removable by a caretaker for disposal. The adhesive may be, for example, the same type that is generally found on Duct Tape®. The adhesive material 6 is only applied to an inner surface 5 of the outer sole layer as shown in FIGS. 2a and 2b.

The peel away layer 7 is any material that easily releases from the adhesive, preferably having a wax coating. The peel away layer bonds lightly to the adhesive while the adhesive strongly bonds to the outer sole member.

The medicine 3 encapsulated within the membrane envelope 2 is any medicine or treatment designed for application to a horses hoof, sole or frog for the treatment of infection, pain or other ailment. It can include antibiotics, salt, drawing salves, pain relievers, poultice, homeopathic medicines, DMSO, silver, antifungal agents, or hoof hardeners. Any other liquid or dry substance such as gel, salve, ointment, paste, powder, or crystals that can be encapsulated within a membrane envelope is acceptable. The bandage of the present invention can also be converted for the treatment of other hoofed animals as well as padded foot animals by varying size and shape of the bandage to compliment different species of animals.

The bandage preferably is made to be disposable and may be distributed for use inside of an enclosed wrapper. The bandage and wrapper can be sterile. Biodegradable materials are also contemplated for this bandage to be environmentally friendly.

A method for delivering a medicine to a horse's hoof according to one aspect of the invention will be described. A membrane envelope bandage is provided. The peel away layer is removed to reveal an adhesive layer. The membrane envelope may be punctured or broken. The membrane envelope is placed on the horse's sole and then the adhesive layer is applied to the horse's hoof wall. The horse is then permitted to stand on the bandage which may rupture the membrane envelope if the caregiver has not already punctured it. Medicine or treatment is then directly delivered to the horse's foot and encased by the outer sole member. An absorbent layer holds the medicine in place and provides cushion for the bottom of the horse's hoof to protect and relieve pain. The outer sole member protects the horse's foot and prevents contamination of the horse's foot.

What is claimed is:

1. A membrane envelope bandage for treating an animal foot or hoof comprising:
an outer sole member having an inner surface and an outer surface for protecting said hoof or foot;
a membrane envelope, wherein said membrane envelope has a dimension that is smaller than said outer sole member;
a medicine or treatment contained within said membrane envelope;
an absorbent layer, wherein said absorbent layer has a dimension that is smaller than said outer sole member, said absorbent layer disposed between said inner surface of said outer sole member and said membrane envelope;
an adhesive layer on said inner surface of said outer sole member for attaching said outer sole member to said hoof or foot.

2. The bandage of claim 1, wherein said outer sole member is elasticized.

3. The bandage of claim 1, further comprising a reinforcement guard disposed on said outer surface of said outer sole member that is the same general shape and size as a bottom surface of a horse's hoof or animal's foot to prevent damage to said bandage and to further protect said horse's hoof or animal's foot.

4. The bandage of claim 1, further comprising a peel away layer disposed on said adhesive layer.

5. The bandage of claim 1, wherein said membrane envelope has a tear line and an opening tab connected to said tear line for opening said membrane envelope.

6. The bandage of claim 1, wherein said outer sole member is generally arcuate in shape and further comprises perforations defining tear away notches, wherein in use, said tear away notches are removed along said perforations to enable said outer sole member to conform to a horse's hoof wall.

7. The bandage of claim 1, wherein said membrane envelope further comprises a partition dividing said membrane envelope into two or more compartments for holding different substances.

8. The bandage of claim 1, wherein said medicine or treatment comprises one or more of antibiotics, salts, drawing salves, pain relievers, poultice, homeopathic medicines, DMSO, silver, antifungal agents, or hoof hardeners.

9. The bandage of claim 1, wherein said medicine or treatment is in the form of a substance comprising of one or more of a liquid, gel, salve, ointment, paste, powder, and crystals.

10. The bandage of claim 1, wherein said absorbent layer has the same general shape of a bottom of said animal's foot.

11. A membrane envelope bandage for treating a horse's hoof comprising:
an outer sole member having an inner surface and an outer surface for protecting said hoof, said outer sole member having a generally arcuate shape to conform to said hoof when applied thereto;
an absorbent layer applied to said inner surface of said outer sole member and having a dimension that is smaller than said outer sole member;
a membrane envelope applied to said absorbent layer;
a medicine or treatment contained within said membrane envelope;
an adhesive layer on an outer periphery edge of said inner surface of said outer sole member for attaching said outer sole member to said hoof.

12. The bandage of claim 1, wherein said membrane envelope is semi-permeable or non-permeable.

13. The bandage of claim 1, further comprising a reinforcement guard integral with said outer sole member that is the same general shape and size as a bottom surface of a horse's or animal's foot to prevent damage to said bandage and to further protect said horse's hoof or animal's foot.

14. The bandage of claim 1, wherein said membrane envelope is attached to said absorbent layer.

15. The bandage of claim 1, wherein said membrane envelope is attached to said inner surface of said outer sole member.

16. A membrane envelope bandage for treating a horse's hoof comprising:
a generally arcuate shaped outer sole member having an inner surface and an outer surface;
a membrane envelope, wherein said membrane envelope has a dimension that is smaller than said outer sole member and not larger than a bottom surface of said horse's hoof;
a medicine or treatment contained within said membrane envelope;
an absorbent layer affixed to said inner surface of said outer sole member, wherein said absorbent layer has a dimension that is smaller than said outer sole member, said absorbent layer disposed between said inner surface of said outer sole member and said membrane envelope;
an adhesive layer on said inner surface of said outer sole member for attaching said outer sole member to a hoof wall of said horse's hoof;
a peel away layer affixed to said adhesive layer;
a reinforcement guard on an outermost surface of said bandage that generally conforms to the shape and size of a bottom surface of a horse's hoof for protecting said bandage from wear.

17. A method for delivering a medicine to an animal foot or hoof comprising:
a) providing the membrane envelope bandage of claim 1;
b) affixing said outer sole member to said animal's foot via said adhesive layer so that said membrane envelope is applied to a bottom surface of said foot,
c) breaking said membrane envelope to deliver said medicine directly to the sole of said animal's hoof or foot.

18. The method of claim 17, wherein said breaking step occurs when said animal bears weight on said foot or hoof.

19. The method of claim 17, wherein said breaking step occurs prior to said affixing step when a caregiver manually opens said membrane envelope to release said medicine or treatment for application to said animal's foot or hoof.

20. A method for delivering a medicine to an animal foot or hoof comprising:
a) providing the membrane envelope bandage of claim 4;
b) peeling off the peel away layer;
c) affixing said outer sole member to said animal's foot via said adhesive layer so that said membrane envelope is applied to a bottom surface of said foot,
c) delivering medicine directly to the sole of said animal's hoof or foot from said membrane envelope.
